(19) United States
(12) Patent Application Publication
(10) Pub. No.: US 2009/0136461 A1
(43) Pub. Date: May 28, 2009

(54) Neuronal differentiation method of adult stem cells using small molecules

(76) Inventors: Moon Suk Kim, Yuseong-gu (KR); Hyun Hee Ahn, Cheongju-si (KR); Jung Hwa Lee, Buk-gu (KR); Hee Jung Jung, Seo-gu (KR); Hai Bang Lee, Yuseong-gu (KR); Kyung Sook Kim, Seo-gu (KR); Ju Young Lee, Changwon-si (KR)

Correspondence Address: FROMMER LAWRENCE & HAUG LLP, 745 FIFTH AVENUE, NEW YORK, NY, 10151, US

(21) Appl. No.: 12/284,117

(22) Filed: Sep. 18, 2008

(30) Foreign Application Priority Data
Nov. 28, 2007 (KR) 10-2007-0122363
Apr. 2, 2008 (KR) 10-2008-0030876

(51) Int. Cl. A61K 35/30; C12N 5/08; A61P 25/00

(52) U.S. Cl. 424/93.7; 435/377

(57) ABSTRACT

The present invention relates to a neuronal differentiation method of adult stem cells using small molecules, more particularly to a method for inducing differentiation of adult stem cells into nerve cells using small molecules, which enables effective differentiation into nerve cells and, thus, is useful in treating intractable CNS disorders such as Parkinson's disease, dementia, Alzheimer's disease and spinal cord injury.

### CROSS-REFERENCE TO RELATED APPLICATION

This application claims priorities under 35 U.S.C. §119 to Korean Patent Application No. 10-2007-0122363, filed on Nov. 28, 2007, and Korean Patent Application No. 10-2008-0030876, filed on Apr. 2, 2008, in the Korean Intellectual Property Office, the contents of which are incorporated herein by reference.

### BACKGROUND

1. Technical Field

The present invention relates to a neuronal differentiation method of adult stem cells using small molecules.

2. Description of the Related Art

Despite the remarkable achievements in medical field, there are still many intractable diseases which cannot be cured with the modern medical science, and CNS (central nervous system) diseases are typical examples. In modern societies, nerve damages caused by industrial disasters and traffic accidents are on the increase. In advanced countries, the increase in social and economic cost due to increased degenerative neuronal diseases has been raised as an important issue. Parkinson's disease, one of the most treatable nervous-system diseases, results from the loss of dopaminergic neurons in the substantia nigra of the midbrain. The disorder is characterized by rigidity of skeletal muscles and it has been estimated that there are about 100,000 patients in Korea [Castano et al., J. Neurochem., 1998, 70:1584-1592]. No effective method had been known to treat the disorder previously. In 1998, however, neural stem cells were successfully isolated and dopamine-secreting cells were differentiated therefrom. Further, when they were transplanted into a Parkinson's disease animal model, they showed good potential to be used in therapeutic treatment [McKay et al., Nat. Neurosci., 1998, 1(4):290-295]. This indicates that stem cells may provide a new opportunity to cure intractable nervous-system diseases.

Stem cells are progenitor cells capable of renewing themselves through numerous cycles of cell divisions and being differentiated into specialized cell types in response to specific cell signals. The stem cells have differential plasticity, or the ability to differentiate into various cells, depending on intrinsic regulatory factors and niche, i.e., extracellular environment [Lee et al., Tissue engineering and regenerative medicine, 2005, 2(3):264-273]. Accordingly, depending on the development stages that affect the differential plasticity, stem cells may be classified into embryonic stem cells (ESCs) found in blastocysts, and adult stem cells found in adult tissues. ESCs are extracted from the inner cell mass (ICM) of blastocysts within 14 days after fertilization. Although they have potent differentiating capacity, they are at the center of ethical debate on the dignity of life and are associated with tumorigenesis problem. Adult stem cells act as a repair system for restoring cell damages resulting from genetic and pathological causes. Although they have limited differentiating capacity as compared to ESC, the adult stem cells can function stably.

Typical adult stem cells that can be utilized to treat nervous-system diseases are neural stem cells. However, because these stem cells exist in specific regions of the brain, such as the subventricular zone (SVZ) and the hippocampus, it is impossible to isolate them in therapeutically sufficient amounts. Bone marrow-derived mesenchymal stem cells, muscle-derived stem cells and adipose-derived stem cells are advantageous in that they exhibit in vitro self-renewing abilities and can be easily isolated and cultured as adult stem cells capable of differentiating into bones, cartilages and adipose tissues under adequate conditions for differentiation. Further, as the stem cells derived from bone marrow, muscles or adipose tissues were reported to have the ability to transdifferentiate into nerve cells, the possibly of utilization thereof as cell source for the treatment of CNS diseases is proposed [Pittenger et al., Science, 1999, 284(2):143-147; Huard et al., Curr. Opin. Biotechnol., 2004, 15(5):419-23]. As a way of improving the applicability of stem cells for the treatment of intractable CNS diseases, there has been introduced a method of introducing specific genes to induce differentiation into nerve cells [Low et al., Cell Mol. Neurobiol., 2007, 27(5):75-85; Kim et al., Eur. J. Neurosci., 2002, 16(10):1829-1838]. However, proteins, i.e., the product of gene expression, in living organisms play more than one function at the same time, and thus an unexpected result may occur when specific genes are removed completely.

Small molecules which selectively bind macromolecules such as proteins and genes, and regulate various biological pathways and signals are good candidates to be used as a drug for the treatment of certain diseases. Accordingly, by using small molecules, it is possible to effectively control the capacity or differentiable properties of transplanted stem cells [Ding et al., Curr. Opin. Chem. Biol., 2007, 11(3):252-230; Schultz et al., Nat. Bitechnol., 2004, 22(7):833-840].

The most commonly used small molecules used to differentiate stem cells into nerve cells are a mixture of dimethyl sulfoxide (DMSO) and butylated hydroxyanisole (BHA, M.W. 180.2). The inducement of differentiation using this mixture resulted in morphological changes and gene expressions characteristic of nerve cells. But, differentiation into glial cells was also observed. In addition to this non-specificity, long-term maintenance of differentiation is not possible due to its strong cell toxicity [Black et al., J. Neurosci. Res., 2000, 61:364-370].

Recently, numerous small molecules including purines, pyrimidines and quinazolines are proposed as strong tools for controlling self renewal and selective differentiation of progenitor cells. For example, differentiation of mesenchymal progenitor cells of mouse into muscle cells using 5-azacytidine-C, a demethylation compound of DNA, was reported [Lassar et al., Cell, 1986, 47(649-656)]. Further, differentiation of neural progenitor cells into nerve cells using a small molecule neuropathiazol was reported [Ding et al., Angewandte Chemie., 2006, 118(4):605-607]. Further, it was reported that transplanted stem cells can restore damaged tissues and facilitate the growth of intrinsic nerve cells in an animal model of nervous-system diseases [Shetty et al., Stem Cells, 2007, 25(8):2014-2017]. However, there have not been many researches conducted on differentiation of adult mesenchymal stem cells into nerve cells using small molecules.

Accordingly, the need of researches on inducement of differentiation of adult mesenchymal stem cells into nerve cells using small molecules is increasing with respect to the treatment of intractable CNS diseases.

The above information disclosed in this Background section is only for enhancement of understanding of the background of the invention and therefore it may contain information that does not form the prior art that is already known in this country to a person of ordinary skill in the art.

### SUMMARY OF THE DISCLOSURE

The inventors of the present invention have completed the present invention by isolating and culturing stem cells derived from bone marrow, muscles and adipose tissues as cell source for the regeneration of the CNS, and confirming their differentiation into nerve cells using small molecules through molecular biological tools.

Accordingly, an object of the present invention is to provide stem cells derived from bone marrow, muscle and adipose tissues as cell source for differentiation into nerve cells.

Another object of the present invention is to provide a method for differentiating stem cells into nerve cells using small molecules.

In an aspect, the present invention is characterized by a method for differentiating adult stem cells into nerve cells using small molecules.

The nerve cells differentiated by the method according to the present invention may be included in a composition useful for the treatment intractable CNS disorders, such as Parkinson's disease, Alzheimer's disease and damage of spinal cord.

In accordance with the present invention, adult stem cells can be differentiated into nerve cells using small molecules. Thus differentiated adult stem cells can be widely used as cell source for the treatment of CNS disorders, such as Parkinson's disease, dementia, Alzheimer's disease and spinal cord injury.

### BRIEF DESCRIPTION OF THE DRAWINGS

The above and other objects, features and other advantages of the present invention will be more clearly understood from the following detailed description taken in conjunction with the accompanying drawings, in which:

FIG. 1 shows inverted microscopic images of (A): bone marrow-derived mesenchymal stem cells, (B): muscle-derived stem cells and (C): adipose-derived stem cells isolated in vitro from bone marrow, skeletal tissues and adipose tissues of 5-week-old Fischer rats, respectively, and subcultured for five generations;

FIG. 2 shows antigens detected on the surface of adult stem cells using FACS analysis (A): antigens expressed on the surface of bone marrow-derived mesenchymal stem cells, (B): antigens expressed on the surface of muscle-derived stem cells and (C): antigens expressed on the surface of adipose-derived stem cells;

FIG. 3 shows inverted microscopic images of stem cells derived from bone marrow, skeletal muscles and adipose tissues, differentiated by treating with 10 μM small molecules (QHA-2 and BHA-1) and 2 μM retinoic acid (A): bone marrow-derived mesenchymal stem cells [A1: QHA-2, A2: BHA-1, A3: retinoic acid as positive control], (B): muscle-derived stem cells [B1: QHA-2, B2: BHA-1, B3: retinoic acid as positive control], and (C): adipose-derived stem cells [C1: QHA-2, C2: BHA-1, C3: retinoic acid as positive control];

FIG. 4 shows images of adipose-derived stem cells differentiated by treating with 10 μM small molecules [A: QHA-2, B: BHA-1, C: BHA-2, D: BHA-3, E: BHA-4, F: AAHA-1, G: AAHA-2, H: KR63240, I: KR63244];

FIG. 5 shows cell toxicity test result of treating bone marrow-derived mesenchymal stem cells with 10 μM and 100 μM QHA-2 [(A) shows microscopic images of cell morphology after treating at concentrations of 10 μM (A1) and 100 μM (A2), and (B) shows MUT assay result];

FIG. 6 shows cell toxicity test result of treating bone marrow-derived mesenchymal stem cells and muscle-derived stem cells with 10 μM QHA-2 and BHA-1 and 2 μM retinoic acid [(A) shows the result for bone marrow-derived mesenchymal stem cells, and (B) shows the result for muscle-derived stem cells];

FIG. 7 shows immunocytochemical staining images of nerve cell markers after differentiating bone marrow-derived mesenchymal stem cells by treating with 10 μM QHA-2 and BHA-1 and 2 μM retinoic acid [(A) shows the result of staining bone marrow-derived mesenchymal stem cells with neuron-specific enolase (NSE) [A1: QHA-2, A2: BHA-1, A3: retinoic acid], and (B) shows the result of staining bone marrow-derived mesenchymal stem cells with beta III tubulin (Tuj1) [B1: QHA-2, B2: BHA-1, B3: retinoic acid];

FIG. 8 shows immunocytochemical staining images of nerve cell markers after differentiating skeletal muscle-derived stem cells by treating with 10 μM small molecules [(A: BHA-2, B: BHA-3, C: BHA-4, D: MHA-1, E: MHA-2). 1 shows the result of staining with nerve cell marker NSE, 2 shows the result of staining with Tuj1, 3 shows the result of staining with astrocyte marker GFAP, and 4 shows the result of staining with oligodendrocyte marker CNPase];

FIG. 9 shows NSE gene expression result for the RNAs isolated from bone marrow-derived mesenchymal stem cells differentiated by treating with 10 μM QHA-2 and BHA-1 and 2 μM retinoic acid, confirmed by RT-PCR; and

FIG. 10 shows NF (neurofilament) gene expression result for the RNAs isolated from muscle-derived stem cells differentiated by treating with 10 μM QHA-2 and BHA-1 and 2 μM retinoic acid, confirmed by RT-PCR.

### DETAILED DESCRIPTION OF THE INVENTION

Hereinafter, reference will be made in detail to various embodiments of the present invention, examples of which are illustrated in the accompanying drawings and described below. While the invention will be described in conjunction with exemplary embodiments, it will be understood that the present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined in the appended claims.

The present invention relates to a method for inducing differentiation of adult stem cells into nerve cells using small molecules which enable effective differentiation into nerve cells and, thus, are effective in treating intractable CNS disorders, such as Parkinson's disease, dementia, Alzheimer's disease and spinal cord injury.

In the first place, description will be given about the adult stem cells used in the present invention.

Stem cells are progenitor cells characterized by the ability to renew themselves through numerous cycles of cell division and the capacity to differentiate into specialized cell types in response to specific cell signals. Due to these characteristics, the stem cells can be used to restore otherwise unregeneratable nerve cells and treat intractable CNS diseases.

Because adult stem cells derived from bone marrow, muscles or adipose tissues have superior self-renewing ability in vitro and can be isolated easily, they can solve the ethical problem of ESC and their ability to differentiate into nerve cells proposes a new way of cell treatment.

Isolation and culturing of these stem cells and surface expressing antigens thereof will be described in detail in Example 1.

Small molecules can be a useful tool for understanding life phenomena through selective differentiation control of cells. Since the completion of genome mapping, genetic manipulation has been applied universally in researches of cell regulation mechanisms. Although it is useful to investigate into functions of specific genes through point mutation or knockout, the genetic manipulation is disadvantageous in that it is irreversible and timely control is difficult. In contrast, small molecules enable reversible and timely control.

Preferably, the small molecules used in the present invention may be, for example, at least one selected from purines, pyrimidines, quinazolines, pyrazines, pyrrolopyrimidines, pyrazolopyrimidines, phthalazines, pyridazines and quinoxalines.

The small molecules used in the present invention may be at least one selected from the group consisting of alkylthiobenzimidazoles, benzhydroxyamides, quinoxaline hydroxyamides and acylaminomethyl hydroxyamides. These compounds are histone deacetylase inhibitors (hereinafter, HDAC inhibitors), which acetylate chromatin and promote the expression of transforming growth factors and the genes essential for the inducement of differentiation, thereby inducing differentiation of tumor genes, inhibiting angiogenesis and, ultimately, exhibiting anticancer activity of destroying tumor cells. Therefore, they are important targets in the development of anticancer drugs [Sausville et al., The Oncologist, 2001, 6:517-537].

Preferably, the small molecules used in the present invention are used at a concentration of 1 nM to 100 μM. If the concentration is below 1 nM, the effect of differentiation is insignificant. And, if it exceeds 100 μM, the compound may crystallize and it may result in cell toxicity. More preferably, the concentration is in the range of from 5 to 30 μM.

The small molecules used in the present invention are alkylthiobenzimidazoles, benzhydroxyamides, quinoxaline hydroxyamides and acylaminomethyl hydroxyamides, which are listed in the following Table 1.

TABLE 1

| Small molecules | Chemical formulas |
|---|---|
| Alkylthio benzimidazoles | 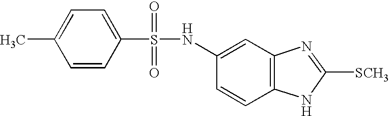 ATBI-1 |
| Benzhydroxyamides | 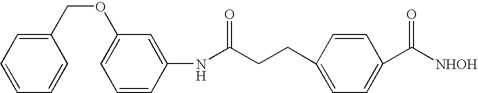 BHA-1 |
| | 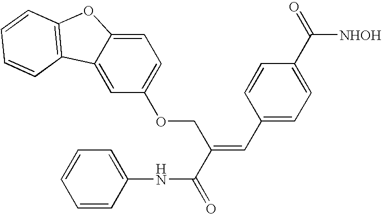 BHA-2 |
| | 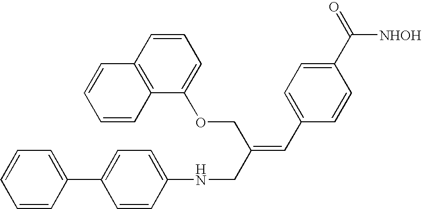 BHA-3 |
| | 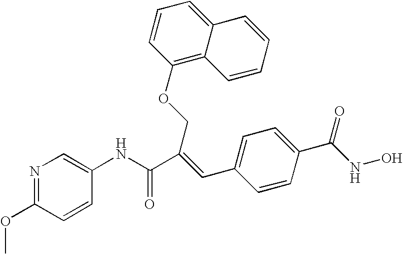 BHA-4 |
| Quinoxaline hydroxyamides | 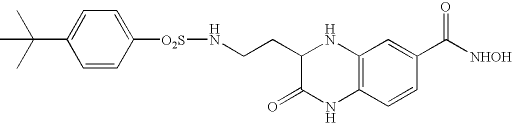 QHA-1 |
| | 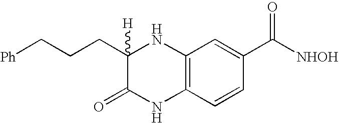 QHA-2 |
| Acylaminomethyl hydroxyamides | 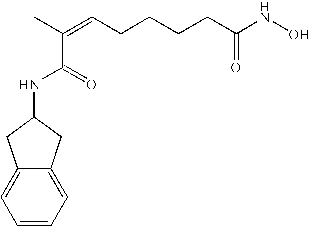 AAHA-1 |
| | 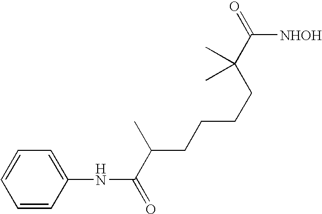 AAHA-2 |

It was confirmed through morphological analysis, immunocytochemical staining and RT-PCR that the above-listed small molecules according to the present invention induce differentiation into nerve cells. It is possible to obtain pure nerve cells by screening out the differentiated stem cells using nerve cell markers. Accordingly, the present invention can provide an effective treatment method for intractable CNS diseases associated with necrosis of nerve cells.

Therefore, the present invention further provides a composition for treating nerve diseases which comprises nerve cells differentiated by the neuronal differentiation method according to the present invention.

As used herein, the nerve diseases refer to CNS disorders such as Parkinson's disease, dementia, Alzheimer's disease and spinal cord injury.

### EXAMPLES

The following examples further illustrate the present invention, but are not intended to limit the scope of the same. In particular, the detailed description about isolation and culturing of stem cells disclosed in the foregoing A Korean Patent Application No. 10-2007-0128788 is incorporated herein by reference in its entirety.

### Example 1

### Isolation and Culturing of Adult Stem Cells

This example illustrates isolation and culturing of stem cells derived from bone marrow, muscles and adipose tissues as cell source for differentiation into nerve cells.

Stage 1: Isolation of Stem Cells

Bone marrow-derived mesenchymal stem cells were isolated as first cell source.

Phosphate buffered saline (Gibco Life Technology, Germany) was perfused into the femur, the fibula and the tibia of Fischer rats weighing 60 to 80 g using a 1 mL syringe. Cells were taken from the hollow interior of the bones and isolated through centrifuge. The cells were cultured using DMEM (Dulbecco's modified Eagle medium; Gibco Life Technology, Germany) containing 10% FBS and 1% antibiotics.

Muscle-derived stem cells were isolated as second cell source.

Skeletal muscle was separated from the femoral region of Fischer rats weighing 60 to 80 g, and cells were isolated using collagenase, trypsin and dispase. The isolated cells were suspended in DMEM containing 5% FBS, 5% horse serum and 2% antibiotics, and distributed to a collagen-coated cell culture flask. 1 hour later, the supernatant was collected from the cell culture flask and subjected to centrifuge. After washing with culture medium, the cells were distributed to a new cell culture flask. At this time, most of the fibroblasts adhered to the bottom of the flask. When the fibroblasts filled about 30 to 40% of the cell culture flask, the supernatant was collected again and subjected to centrifuge. Then, after washing with culture medium, the cells were distributed to a new cell culture flask. 2 hours, 1 day, 2 days and 3 days later, the same procedure was repeated to isolate muscle-derived stem cells.

Adipose-derived stem cells were isolated as third cell source.

Visceral adipose was separated from Fischer rats weighing 60 to 80 g, and cells were isolated after treatment with collagenase. The cells were cultured using DMEM containing 10% FBS and 1% antibiotics.

Stage 2: Culturing of Stem Cells

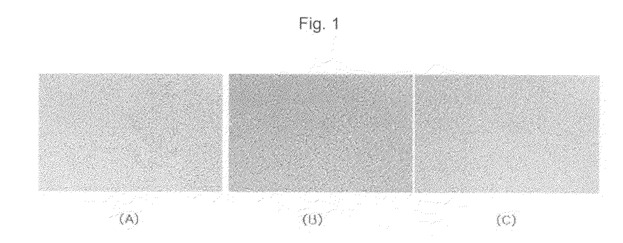

The stem cells isolated in Stage 1 were distributed to a culture flask at a concentration of 10³ to 10⁴ cells/cm², and cultured in 37° C., 5% CO₂ incubator. The culture medium was replaced once in 3 days. When the cells grew to fill 70% or more of the culture flask, they were prepared into single cells by treating with 0.05% trypsin for 5 minutes, and subjected to subculturing [FIG. 1].

Stage 3: Confirmation of Stem Cell Surface Antigens

The stem cells isolated in Stage 1 were prepared into single cells by treating with 0.05% trypsin and washed twice with phosphate buffered saline. The respective cells were antibody treated with hematopoietic stem cell marker CD45 (Chemicon, Temecula, Calif.) and mesenchymal stem cell marker CD44 (Chemicon, Temecula, Calif.) at 4° C. for 30 minutes. After washing three times with phosphate buffered saline followed by buffering by adding 30 μL of phosphate buffered saline, antigens expressed on the surface of the stem cells were confirmed using a FACS (BD Biosciences, San Jose, Calif.) analyzer.

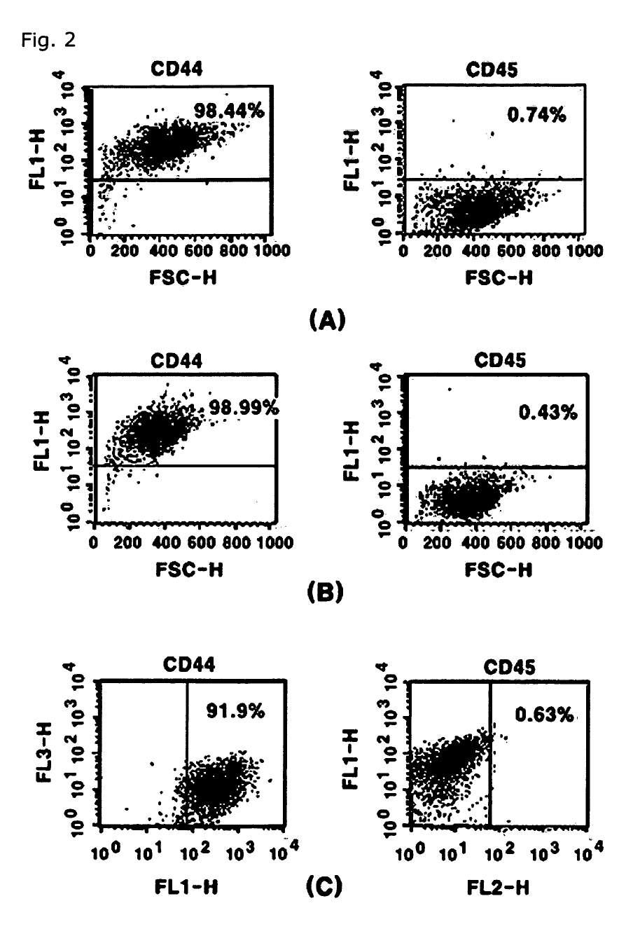

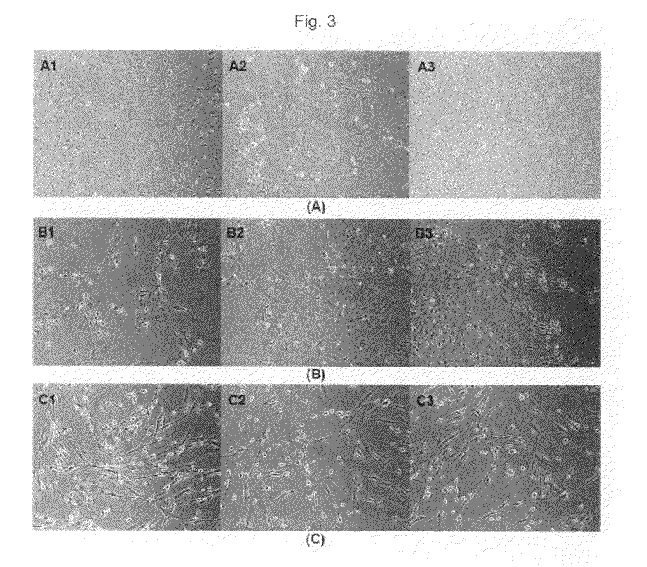

As a result, CD44 expression of over 98% and CD45 expression less than 1% were confirmed. Also, isolation of pure mesenchymal stem cells was confirmed [FIG. 2].

### Example 2

### Differentiation of Stem Cells into Nerve Cells Using Small Molecules

In this example, differentiation of the adult stem cells isolated in Example 1 into nerve cells was induced.

Bone marrow-derived mesenchymal stem cells subcultured for 5 generations were distributed on a well plate. One day later, the cells were treated with DMEM containing 20% FBS and 10 ng/mL b-FGF for a day, so that the cells could proliferate sufficiently. In order to induce differentiation into nerve cells, the cells were treated with differentiation medium containing the small molecules listed in Table 1. The small molecules were used after being dissolved in DMSO (Sigma, USA). The concentration of DMSO was less than 2% of the entire culture medium, and was diluted so that the small molecules were included with a concentration in the range from 1 μM to 100 μM. As negative control, DMEM containing 10% FBS and 1% penicillin-streptomycin was used. And, retinoic acid as positive control, which is a well-known inducer of differentiation into nerve cells, was used after being diluted to 2 μM in DMEM. Differentiation of muscle-derived stem cells and adipose-derived stem cells into nerve cells was induced similarly as in the bone marrow-derived stem cells.

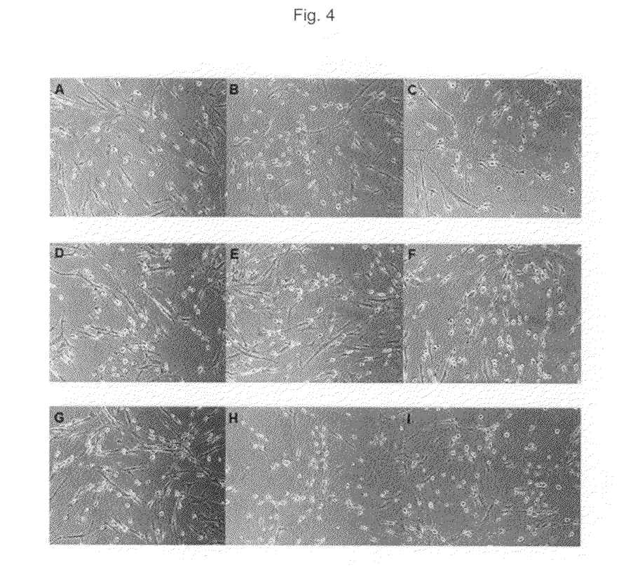

As a result, condensation of cytoplasm and formation of neurites were identified as in nerve cells [FIG. 3 and FIG. 4].

### Example 3

### Evaluation of Toxicity of Small Molecules to Stem Cells

In this example, the toxicity of the small molecules to the stem cells during the differentiation of the adult stem cells into nerve cells in Example 2 was evaluated.

MTT assay is a technique based on the principle that yellow, water-soluble MTT tetrazolium is reduced to purple, water-insoluble MTT formazan by the action of mitochondrial dehydrogenase. The formazan concentration is indicative of the concentration of living and actively metabolizing cells. For MTT assay, bone marrow- and muscle-derived stem cells were distributed to a 24-well plate, at a concentration of 3×10⁴ cells/well, and cultured in an incubator for a day. After treating with culture medium, as in the procedure of inducement of differentiation into nerve cells in Example 2, the culture medium was replaced by 1 mL of new culture medium on day 1 and day 4.

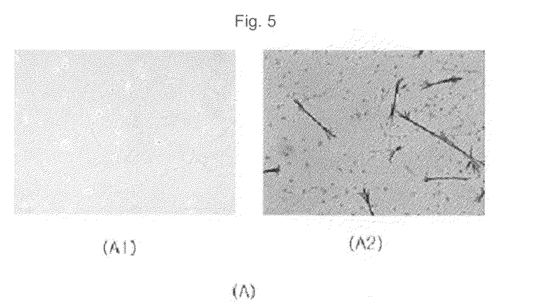

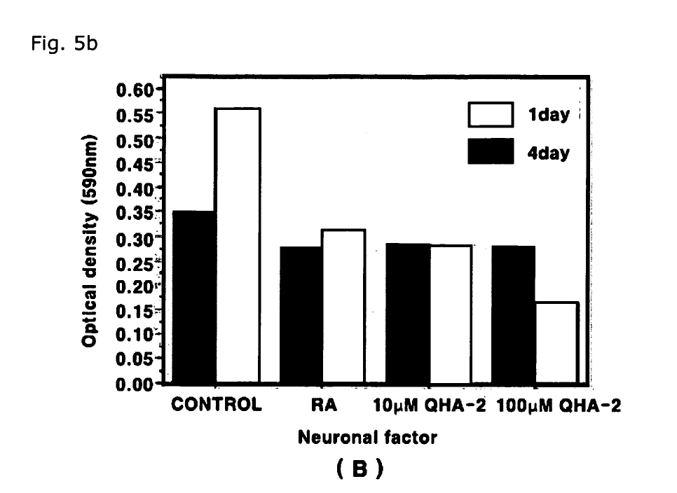

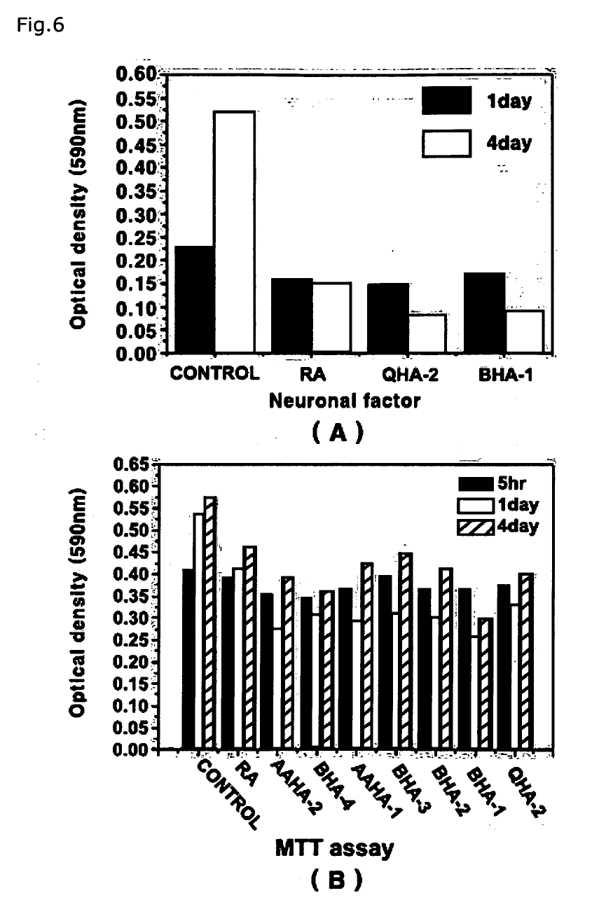

First, cell toxicity was evaluated at concentrations of 2 μM, 10 μM and 100 μM [FIG. 5]. Then, each 100 μL of 5 mg/mL MTT (3-[4,5-dimethylthiazol-2-yl]-2,5-diphenyltetrazolium bromide) solution was added, and the cells were cultured for 4 hours in a 37° C. incubator. When violet crystal was formed, the culture medium and the MTT solution were removed, and stirring was carried out for 30 minutes after adding 1 mL of DMSO solution until the crystal was completely dissolved. After distributing each 100 μL of sample to a 96-well plate, absorbance was measured at 590 nm using an ELISA plate reader (E-max, Molecular Device, USA) [FIG. 6].

### Example 4

### Confirmation of Differentiation into Nerve Cells Using Immunocytochemical Staining

In this example, the expression of nerve cell markers Tuj1 and NSE, astrocyte marker GFAP and oligodendrocyte marker CNPase by the adult stem cells differentiated using the small molecules in Example 2 was confirmed.

Immunocytochemical staining is a technique of identifying proteins expressed by cells, using antibodies.

First, the cells were fixed by treating with 4% paraformaldehyde (Sigma, USA) for 20 minutes, and washed twice with phosphate buffered saline. After inhibiting peroxidase in the cells by treating with 3% hydrogen peroxide for 10 minutes, the cells were washed twice with phosphate buffered saline. After treating with 1% bovine serum albumin (BSA) for 30 minutes and with primary antibodies diluted at 1:100 (Tuj1; Chemicon, Temecula, Calif.) and 1:20 (NSE; Serotec, Oxford, UK) for 1 hour and 30 minutes, the cells were washed twice with phosphate buffered saline. After treating with biotin-bound secondary antibodies for 20 minutes, the cells were washed twice with phosphate buffered saline. After treating with streptavidine for 30 minutes followed by washing twice with phosphate buffered saline, coloring was confirmed with DAB and counterstaining was carried out using hematoxylin. For fluorescent immunostaining, the differentiated cells were fixed using 4% paraformaldehyde (Sigma, USA), followed by washing twice with phosphate buffered saline, treating with 1% BSA for 30 minutes and then treating with primary antibodies diluted at 1:100 (Tuj1; Chemicon, Temecula, Calif.), 1:20 (NSE; Serotec, Oxford, UK), 1:300 (GFAP; Sigma Chemicals, UK) and 1:100 (CNPase; Sigma Chemicals, UK) at 4° C. for 16 hours. After washing twice with phosphate buffered saline followed by treating with secondary antibodies diluted at 1:1000 (rat anti-mouse Alexa Fluor 594; Invitrogen) for 3 hours, counterstaining was carried out using DAPI (4′,6′-diamidino-2-phenylindole).

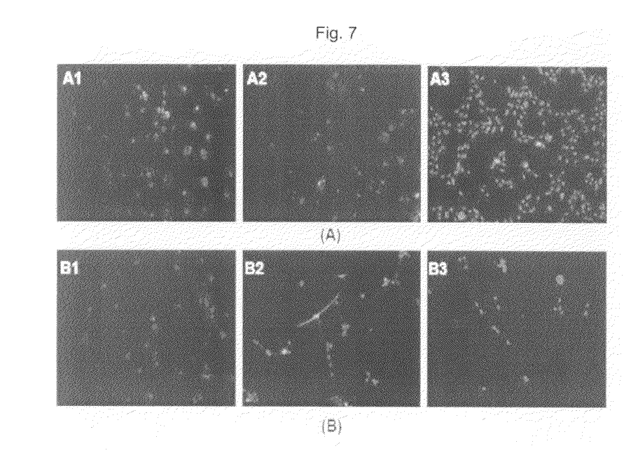

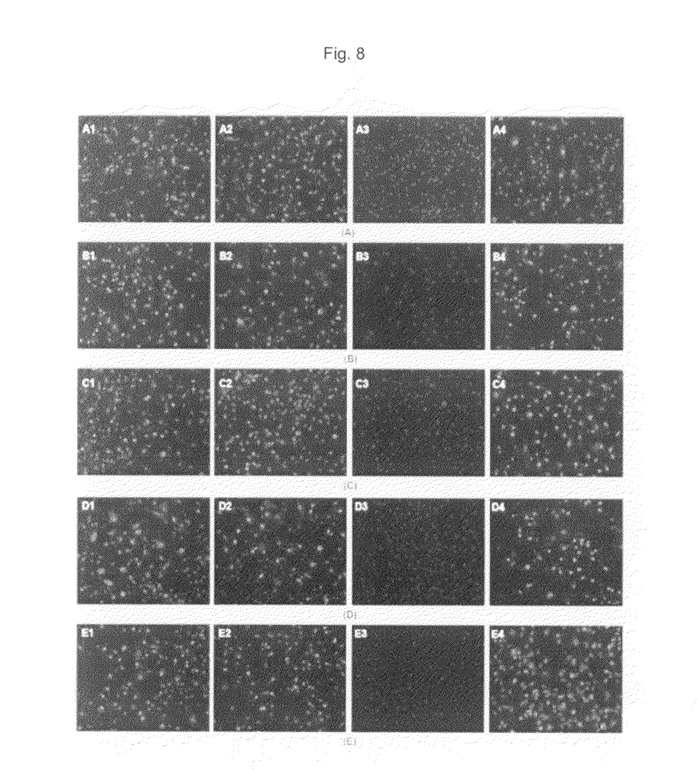

As a result, the expression of nerve cell markers Tuj1 and NSE was identified in the differentiated stem cells. The same result was attained in the positive control group of retinoic acid. Accordingly, the differentiation into nerve cells was confirmed [FIG. 7]. Further, the differentiation into nerve cells could be confirmed with a fluorescence microscope [FIG. 8].

### Example 5

### Confirmation of Differentiation into Nerve Cells Using RT-PCR

In this example, the expression of neuronal genes by the adult stem cells differentiated using the small molecules in Example 2 was confirmed.

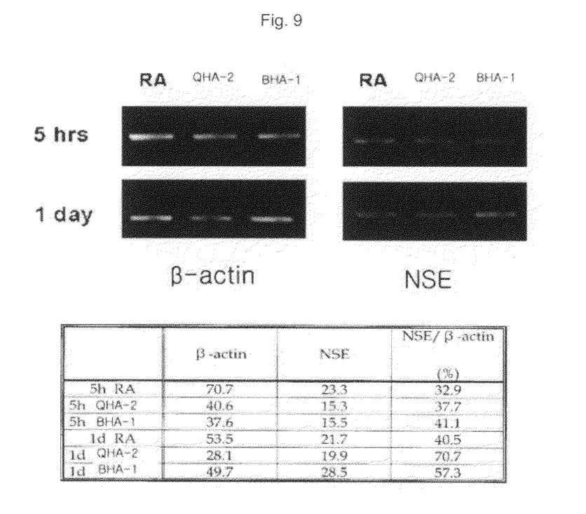

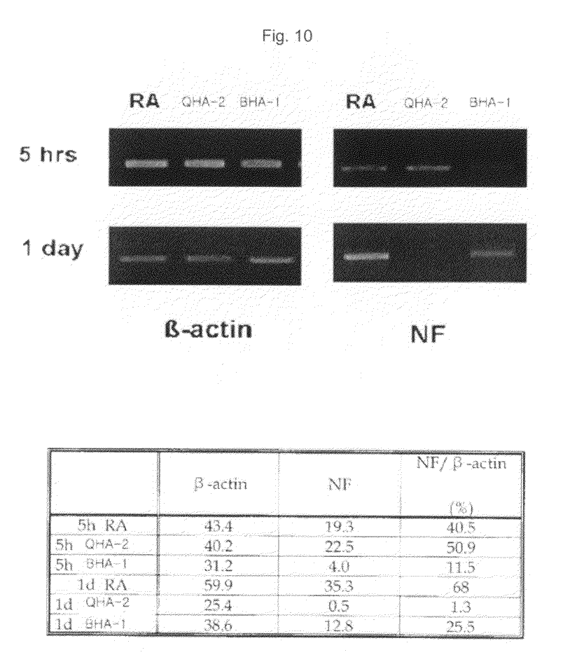

RT-PCR (reverse transcriptase polymerase chain reaction) is a technique for transforming RNAs expressed by cells into cDNAs through reverse transcription, followed by selectively amplifying specific genes through PCR. With this technique, it is possible to confirm the expression of neuronal genes by the differentiated adult stem cells. In order to carry out RT-PCR, the RNAs expressed by the cells were isolated purely using a kit (Qiagen, Germany). The experimental procedure was followed according to the instructions described in the manufacturer's manual. The isolated RNAs were quantized (NanoDrop Technologies, Wilmington, Del.), and RNAs with the value ranging from 1.6 to 1.9 were used. With the isolated RNA as template, cDNAs were prepared through reverse transcription. PCR was carried out using β-actin, NSE and NF as primers to analyze expression of genes. As a result, it was confirmed that the borne marrow-derived mesenchymal stem cells [FIG. 9] and the muscle-derived stem cells [FIG. 10] treated with the small molecules differentiated into nerve cells.

Although the preferred embodiments of the invention have been disclosed for illustrative purposes, those skilled in the art will appreciate that various modifications, additions and substitutions are possible, without departing from the scope and spirit of the invention as disclosed in the accompanying claims.

1. A method for differentiating adult stem cells into nerve cells using a neural inducer, wherein the neural inducer is small molecules.

2. The method according to claim 1, wherein the adult stem cells are derived from bone marrow, skeletal muscle or adipose.

3. The method according to claim 1, wherein the small molecules are small molecules belonging to histone deacetylase inhibitors (HDAC inhibitor).

4. The method according to claim 3, wherein the small molecules belonging to the HDAC inhibitor are at least one selected from alkylthiobenzimidazoles, benzhydroxyamides, quinoxaline hydroxyamides and acylaminomethyl hydroxyamides.

5. The method according to claim 1, wherein the small molecules are used at a concentration of 1 nM to 100 μM.

6. A composition for treating nerve diseases which comprises nerve cells differentiated by the method according to claim 1.

7. The composition as set forth in claim 6, wherein the nerve diseases are CNS (central nervous system) disorders such as Parkinson's disease, dementia, Alzheimer's disease or spinal cord injury.

8. A composition for treating nerve diseases which comprises nerve cells differentiated by the method according to claim 2.

9. A composition for treating nerve diseases which comprises nerve cells differentiated by the method according to claim 3.

10. A composition for treating nerve diseases which comprises nerve cells differentiated by the method according to claim 4.

11. A composition for treating nerve diseases which comprises nerve cells differentiated by the method according to claim 5.
